(19) United States
(12) Patent Application Publication
(10) Pub. No.: US 2004/0111042 A1
(43) Pub. Date: Jun. 10, 2004

(54) Bioelectric telemetering system and method

(76) Inventors: Imre Szabo, Pecs (HU); Kalman Mathe, Pecs (HU); Andre Fenton, New York, NY (US)

Correspondence Address: THE FIRM OF KARL F ROSS, 5676 RIVERDALE AVENUE, PO BOX 900, RIVERDALE (BRONX), NY, 10471-0900, US

(21) Appl. No.: 10/314,890

(22) Filed: Dec. 9, 2002

(51) Int. Cl. A61B 5/04

(52) U.S. Cl. 600/544

(57) ABSTRACT

Bioelectrical activity in a living animal subject is monitored by detecting the bioelectrical signals at a number of points multiplexing the electrical signals and transmitting a multiplexed stream by cellular telephone circuitry. The received data stream is then demultiplexed and monitored to allow the activity at the points on the animal subject to be monitored.

### FIELD OF THE INVENTION

Our present invention relates to a bioelectric telemetering system and method.

### BACKGROUND OF THE INVENTION

In the past for the measurement of biological signals, it has been the pattern to provide bioelectrical transducers at the site at which the biological signals are generated and to connect those transducers by cables to recording and analyzing circuits. Animal studies have been widely carried out with this form of bioelectrical signal transmission and this approach has also been applied to neurological signal transmission in monitoring of the brains of laboratory animals or animal test subjects.

An obvious disadvantage of this approach is that it is not able to transmit signals from free-running or untethered subjects. This may affect the neurological response and may complicate the study.

### OBJECTS OF THE INVENTION

It is, therefore, the principal object of the present invention to provide an improved method of monitoring biological states in animal subjects whereby this drawback is avoided.

Another object of the invention is to provide a method of monitoring biological signals in animal subjects which are not limited by cable tethers of the animal and nevertheless can ensure a high reliability and versatility of signal evaluation.

Yet another object of this invention is to provide a telemetering system for an animal subject which minimizes the physical inhibition of the animal subjects.

### SUMMARY OF THE INVENTION

These objects are attained, in accordance with a feature of this invention, by providing an audio frequency pickup for bioelectrical signals at a site on the animal subjects to be monitored, multiplexing that signal with other similar signals from corresponding sites, transmitting the signal by radio transmission and, particularly, by cellular telephone radio transmission to a location spaced from the animal subject, demultiplexing the signal and thereafter processing the signal at least in part by recording and displaying the signal individual to the original transducer or signal source.

According to a feature of the invention, two such signals are fed to a two-channel audio input for an analog-to-digital converter, e.g. a 24 bit ADC, which digitizes and multiplexes the two analog inputs into a single digital data stream.

The signal is picked up by the receiver which can demultiplex the digital channel for digital signal processing and storage.

The digital signal can also be converted back to an analog signal (by one or more digital-to-analog converters) (DAC) for recording.

Through the use of cellular telephone technology, the audio channels of the cellular telephone can be provided with the inputs and can be supplied to the transmitter of the cellular telephone circuit which can communicate over any required distance with the receiver. The cellular telephone transmitter and audio input system are miniaturized already in cellular telephone technology and thus can be easily mounted on the animal subject or implanted in the animal subject.

The power for driving the unit on the animal may be supplied exclusively by battery, although it has been found to be advantage to utilize both battery power and batteries for driving the cellular transmitter.

We have used the term “cellular transmitter” to refer to that portion of the electronics which comprises the audio signal input and radiofrequency transmitter portion of a cellular telephone which is used to transmit the signal to the receiving station.

The invention also comprises a method of monitoring neural activity which comprises:

(a) connecting electrodes to neural activity points of an animal subject and obtaining analog signals representing activity at the points;

(b) securing to the animal subject a cellular telephone apparatus having audio analog-to-digital converters capable of digitizing and multiplexing two analog inputs each into respective single digital data streams and a gigahertz radiofrequency transmitter for the data streams and connecting the audio analog-to-digital converters to the electrodes, whereby digital data streams representing digitized and multiplexed signals from the electrodes are transmitted by the apparatus;

(c) at a receiver separated from the animal subject receiving the digital data streams transmitted by the apparatus, demultiplexing channels of the received digital data streams to produce digital channels corresponding to the analog inputs, and converting the digital channels to analog outputs; and

(d) recording the analog outputs in a multichannel analog recorder having recording channels assigned to the respective electrodes.

The apparatus can comprise:

a multiplicity of electrodes connected to neural activity points of an animal subject and obtaining analog signals representing activity at the points;

a cellular telephone apparatus mounted on the animal subject and having audio analog-to-digital converters connected to the electrodes and each capable of digitizing and multiplexing two analog inputs each into respective single digital data streams, and a gigahertz radiofrequency transmitter for the data streams and connected to the audio analog-to-digital converters, whereby digital data streams representing digitized and multiplexed signals from the electrodes are transmitted by the apparatus;

a receiver separated from the animal subject receiving the digital data streams transmitted by the apparatus, and provided with a demultiplexer for demultiplexing channels of the received digital data streams to produce digital channels corresponding to the analog inputs, and digital-to-analog converters for converting the digital channels to analog outputs; and

a multichannel analog recorder connected to the receiver and having recording channels assigned to the respective electrodes.

### BRIEF DESCRIPTION OF THE DRAWING

The above and other objects, features, and advantages will become more readily apparent from the following description, reference being made to the accompanying drawing in which:

FIG. 1 is an elevational view showing an animal equipped with the transmitter portion of the device of the invention;

FIG. 2 is a diagram showing the circuitry attached to the skull of the animal;

FIG. 3 is a perspective view showing the circuitry provided with a battery for the transmitter portion;

FIG. 4 is a diagram showing a solar cell attached to the unit;

FIG. 5 is a circuit diagram of an input circuit for reducing common mode noise;

FIGS. 6 and 7 are graphs of pulses for explanation of the invention;

FIG. 8 is a diagram of the overall system incorporating two embodiments of the transmitter stage;

FIG. 9 is a block diagram of the transmitter stage in accordance with one of the latter modes; and

FIG. 10 is a circuit diagram of the digitizing and multiplexing part of the transmitter of the invention.

### SPECIFIC DESCRIPTION

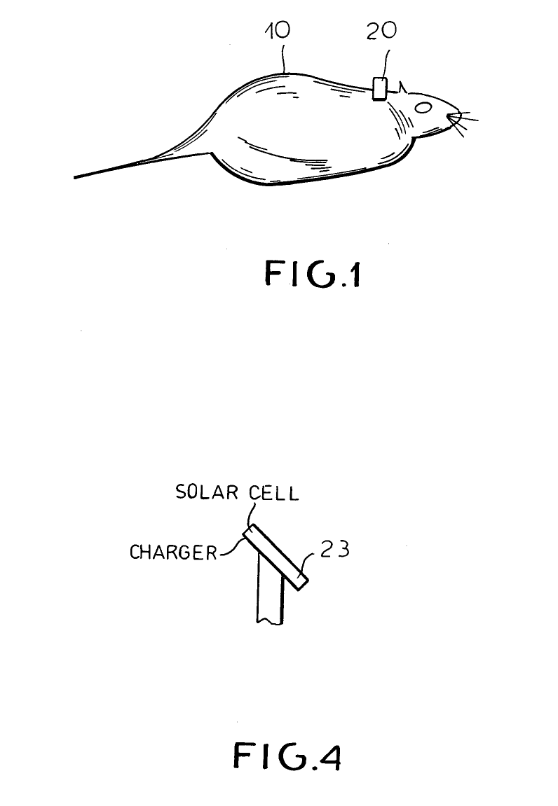

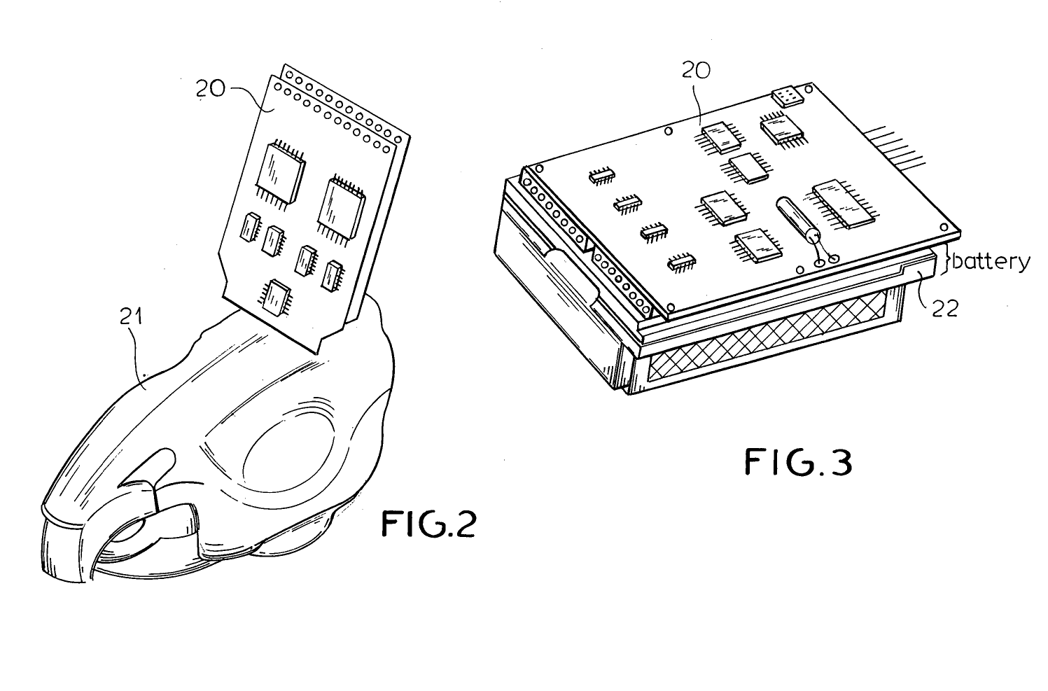

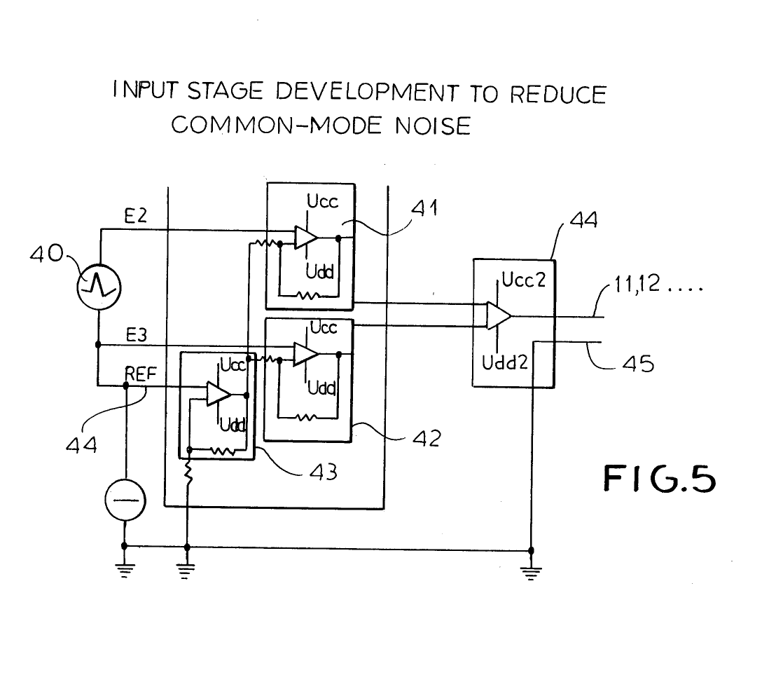

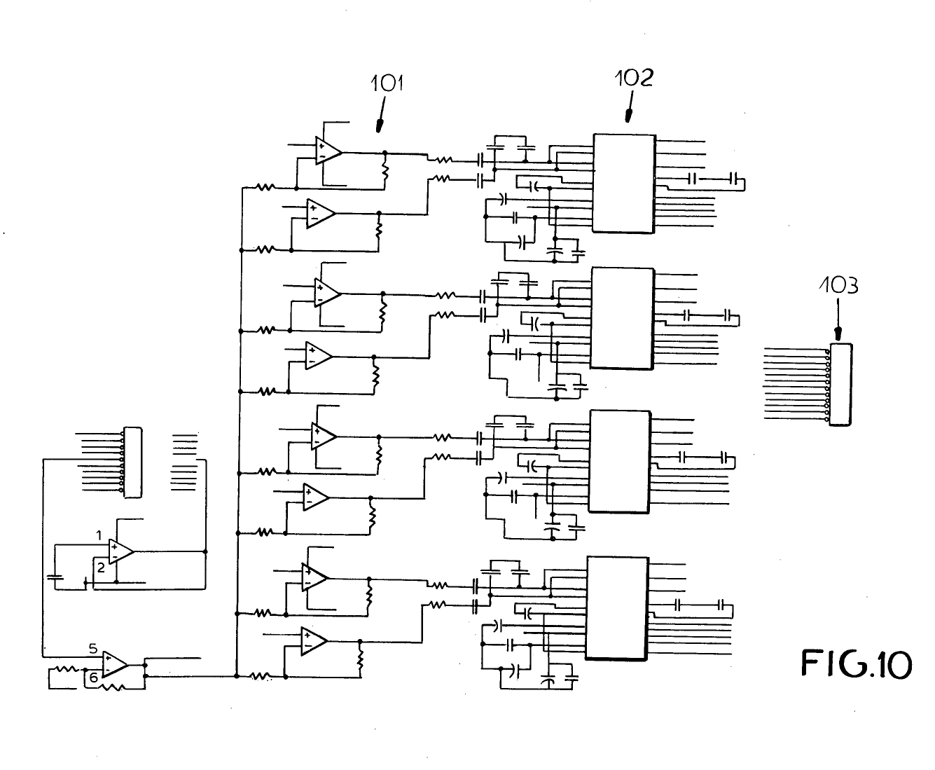

Essentially the present invention utilizes cellular telephone technology to effect telemetering of inputs from a live animal 10 (FIG. 1), here shown as a rat, for hippocampal or other studies, utilizing electrodes 11, 12, 13, 14, 15, 16, 17 and 18 (FIG. 9) which can be implanted in the brain of the animal as represented by the transmitter unit 20 affixed to the skull 21 of the animal in FIG. 2. That transmitter circuit 20 may have a battery 22 suitable for use in a cellular telephone and can also be equipped with a solar cell 23 containing a charger for the battery (FIG. 4). In FIG. 9, the power supply 24 has been shown to include the battery 22 and the solar cell 23.

Basically, the transmitter unit 20 (FIG. 9) can include a 2:1 two-channel 24 bit analog to digital converter and multiplexer 30, 31, 32, 33 for combining the two analog inputs 11, 12, etc. into a single digital data stream which is delivered via line 34, 35, 36, 37 to a 4:1 multiplexer 38. The digital output is conveyed to a gigahertz cell phone transmitter 39 and its antenna 40.

As can be seen from FIG. 5, the inputs 11 and 12 may each derive from electrodes such as the pair of electrodes E2 and E3 and any bioelectrical transducer represented generically at 40 in FIG. 5 which also shows a circuit for reducing the common mode noise. The circuit comprises operational amplifiers 41, 42 and 43, the latter being connected in a follower mode for a reference line 44. The outputs from the operational amplifier 41 and 42 are applied to an output operational amplifier 44 which has both the signal output 11, 12, etc. depending upon the input source, and a ground 45 which can be common to the entire system.

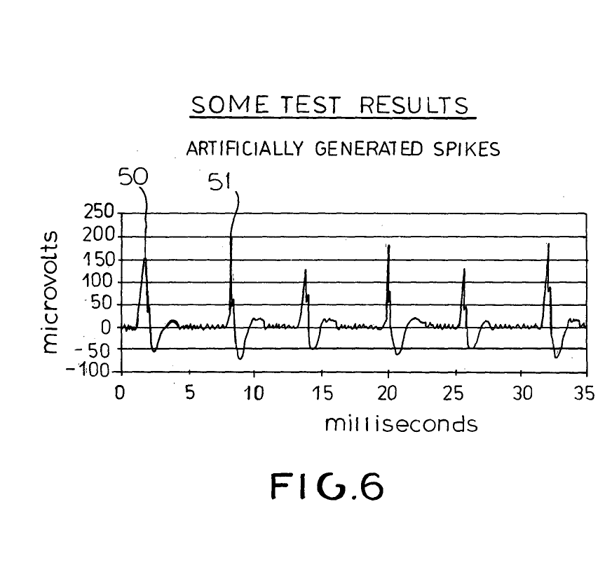

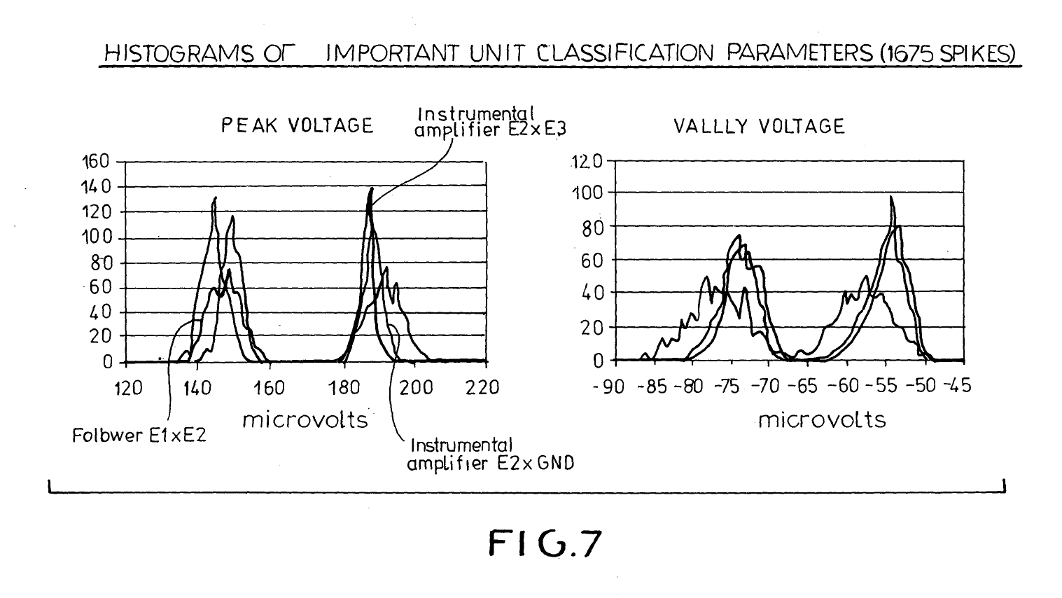

The signals which are received may be spikes as shown at 50 and 51 in FIG. 6 in which artificially generated spikes have been plotted in microvolts along the ordinate against milliseconds along the abscissa. It is possible for an accumulation of spikes to be analyzed utilizing histo-grams as shown in FIG. 7.

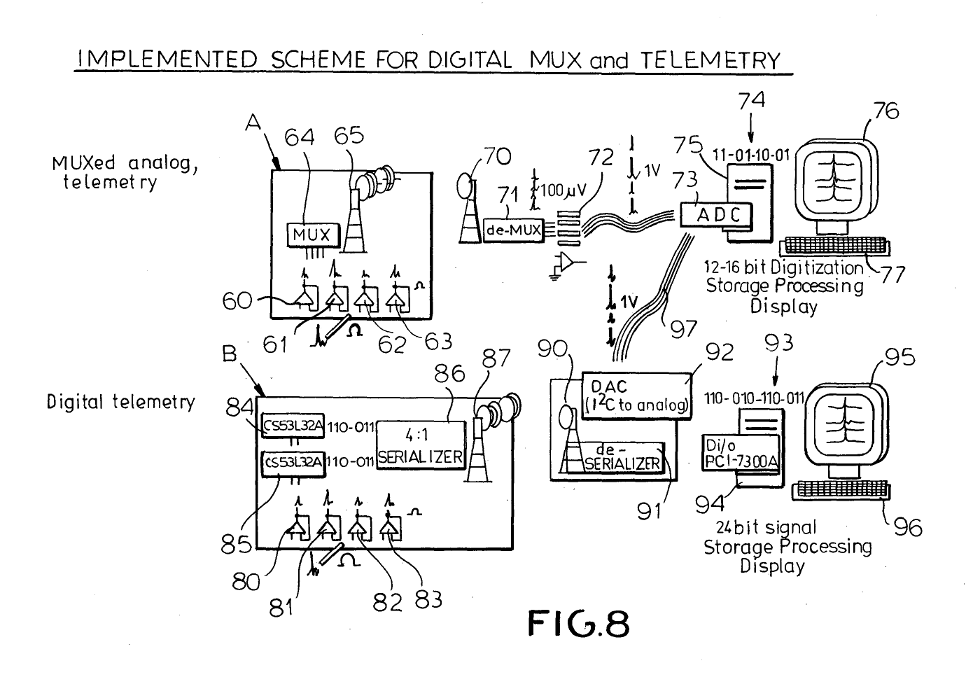

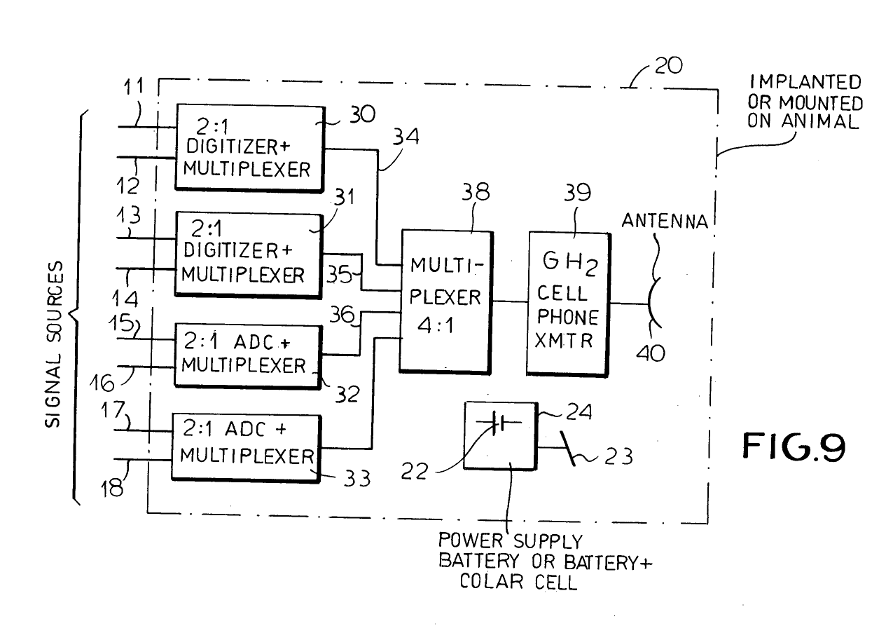

In FIG. 8, the overall system has been shown in greater detail. FIG. 8 shows two modes A and B. In the transmitter mode A, four pulse outputs from circuits of the type shown in FIG. 5 and represented diagrammatically at 60, 61, 62 and 63 are provided as analog inputs to the multiplexer 64 which feeds its output to the GHz cell phone transmitter 65 which transmits to a receiver 70. The latter is connected to the demultiplexer 71 which reforms the four channels at 72 and delivers those channels to a digitizer 73 in the form of an analog to digital converter which can be connected to a digital processing unit 74 including a recorder 75, a display 76 and an input peripheral 77, e.g. a keyboard.

The second mode illustrated at B includes the signal processors 80, 81, 82 and 83 of FIG. 5 and the two channel analog to digital converters 84, 85 etc. which are analogous to those of FIG. 9 providing inputs to the 4:1 serializer or multiplexer 86 which is equivalent to the multiplexer shown at 38 in FIG. 9. The latter feeds the cell phone transmitter 87 which transmits the digital output to the receiver 90 having a deserializer 91 and, if desired, a digital to analog converter (DAC) 92. The deserializer can provide a direct digital output to the digital storage and processing unit 93 which can include a recorder 94, display 95 and keyboard 96. When a digital to analog converter is included at the receiving station, the analog channels may be supplied at 97 to the ADC 73 previously described.

FIG. 10 shows part of the system of FIG. 9 in greater detail and in this illustration the inputs derive from original amplifiers 101 and are fed to two channel multiplexers and digitizers 102 whose outputs, in turn, are supplied to the 4:1 multiplexer 103. The latter is connected, of course, to the cellular telephone transmitter.

While an 8 channel system has been described, a 16 channel system can be used simply by duplicating the circuit of FIG. 9 and feeding the outputs from the two 4:1 multiplexer to an additional multiplexer or by feeding the outputs from the 2:1 ADC multiplexers to an 8 channel multiplexer.

Because of the use of cellular telephone technology, the system described is of simple construction and circuitry and can be used for bioelectric signal telemetering for untethered live animals without significant restriction of mobility.

We claim:

1. A method of monitoring neuronal activity comprising the steps of:
(a) connecting electrodes to neural activity points of an animal subject and obtaining analog signals representing activity at said points;
(b) securing to said animal subject a cellular telephone apparatus having audio analog-to-digital converters capable of digitizing and multiplexing two analog inputs each into respective single digital data streams and a gigahertz radiofrequency transmitter for said data streams and connecting said audio analog-to-digital converters to said electrodes, whereby digital data streams representing digitized and multiplexed signals from said electrodes are transmitted by said apparatus;
(c) at a receiver separated from said animal subject receiving the digital data streams transmitted by said apparatus, demultiplexing channels of the received digital data streams to produce digital channels corresponding to the analog inputs, and converting said digital channels to analog outputs; and
(d) recording said analog outputs in a multichannel analog recorder having recording channels assigned to the respective electrodes.

2. The method defined in claim 1, further comprising the step of multiplexing the single digital data streams supplied to said gigahertz radio frequency transmitter.

3. The method defined in claim 2, further comprising the step of processing the received digital data stream of step (c) to monitor the activity at said points.

4. An apparatus for monitoring neuronal activity comprising:
a multiplicity of electrodes connected to neural activity points of an animal subject and obtaining analog signals representing activity at said points;
a cellular telephone apparatus mounted on said animal subject and having audio analog-to-digital converters connected to said electrodes and each capable of digitizing and multiplexing two analog inputs each into respective single digital data streams, and a gigahertz radiofrequency transmitter for said data streams and connected to said audio analog-to-digital converters, whereby digital data streams representing digitized and multiplexed signals from said electrodes are transmitted by said apparatus;
a receiver separated from said animal subject receiving the digital data streams transmitted by said apparatus, and provided with a demultiplexer for demultiplexing channels of the received digital data streams to produce digital channels corresponding to the analog inputs, and digital-to-analog converters for converting said digital channels to analog outputs; and
a multichannel analog recorder connected to said receiver and having recording channels assigned to the respective electrodes.

5. The apparatus defined in claim 4 wherein each of said electrons is connected to an operational amplifier common-mode voice-reduction stage.

6. The apparatus defined in claim 5, further comprising at least one further multiplexer for multiplexing a plurality of said signal digital data streams prior to transmission by said apparatus.

7. The apparatus defined in claim 6, further comprising a digital signal processor receiving said digital data streams for monitoring the neural activity at said points.

8. A method of monitoring bioelectrical activity in a living animal subject comprising the steps of:
(a) connecting electrodes to points of a living animal subject having bioelectrical activity and obtaining analog electrical signals representing said activity at said points;
(b) multiplexing electrical signals obtained at said points and transmitting a data stream represented by the multiplexed signals through a cellular telephone circuit to a receiver;
(c) demultiplexing a received data stream at said receiver; and
(d) monitoring the activity at said points based upon the demultiplexed data stream.
